SEQUENCE LISTING (52 sequences)

SEQ ID NO: 1 - protein, 9 aa
HLGNVKYLV

SEQ ID NO: 2 - protein, 9 aa
LLMDCSGSI

SEQ ID NO: 3 - protein, 9 aa
GIAGGLALL

SEQ ID NO: 4 - protein, 9 aa
ILYISFYFI

SEQ ID NO: 5 - protein, 9 aa
YISFYFILV

SEQ ID NO: 6 - protein, 9 aa
GIYKELEDL

SEQ ID NO: 7 - protein, 9 aa
HIFDGDNEI

SEQ ID NO: 8 - protein, 9 aa
YLKTIQNSL

SEQ ID NO: 9 - protein, 9 aa
YLQKIQNSL

SEQ ID NO: 10 - protein, 9 aa
YLQKIKNSL

SEQ ID NO: 11 - protein, 9 aa
YLNKIQNSL

SEQ ID NO: 12 - protein, 9 aa
LRKPKHKKL

SEQ ID NO: 13 - protein, 9 aa
LKKIKNSIS

SEQ ID NO: 14 - protein, 9 aa
QVRIKPGSA

SEQ ID NO: 15 - protein, 9 aa
ANKPKDGLD

SEQ ID NO: 16 - protein, 9 aa
ASKNKEKAL

SEQ ID NO: 17 - protein, 9 aa
KNKEKALII

SEQ ID NO: 18 - protein, 9 aa
MPNDPNRNV

SEQ ID NO: 19 - protein, 9 aa
MPNYPNRNV

SEQ ID NO: 20 - protein, 9 aa
MPNNPNRNV

SEQ ID NO: 21 - protein, 9 aa
KPIVQYDNF

SEQ ID NO: 22 - protein, 9 aa
IPSLALMLI

SEQ ID NO: 23 - protein, 9 aa
MPLETQLAI

SEQ ID NO: 24 - protein, 10 aa
NPDPNANPNV

SEQ ID NO: 25 - protein, 10 aa
NPENPPNPDI

SEQ ID NO: 26 - protein, 9 aa
IPDSIQDSL

SEQ ID NO: 27 - protein, 9 aa
EPAPFDETL

SEQ ID NO: 28 - protein, 9 aa
GPFMKAVCV

SEQ ID NO: 29 - protein, 9 aa
LSVSSFLFV

SEQ ID NO: 30 - protein, 10 aa
GSANKPKDEL

SEQ ID NO: 31 - protein, 8 aa
CSSVFNVV

SEQ ID NO: 32 - protein, 8 aa
NSEKDEII

SEQ ID NO: 33 - protein, 9 aa
GSSNSRNRI

SEQ ID NO: 34 - protein, 9 aa
VSQTNFKSL

SEQ ID NO: 35 - protein, 9 aa
KSLLRNLGV

SEQ ID NO: 36 - protein, 9 aa
QSDSEQERL

SEQ ID NO: 37 - protein, 9 aa
RTKASKETL

SEQ ID NO: 38 - protein, 8 aa
HTLETVNI

SEQ ID NO: 39 - protein, 9 aa
ISDVNDFQI

SEQ ID NO: 40 - protein, 8 aa
ISKYEDEI

SEQ ID NO: 41 - protein, 9 aa
ISAEYDDSL

SEQ ID NO: 42 - protein, 8 aa
KSLYDEHI

SEQ ID NO: 43 - protein, 9 aa
LSEDITKYF

SEQ ID NO: 44 - protein, 7 aa
TKYFMKL

SEQ ID NO: 45 - protein, 11 aa
KTASCGVWDEW

SEQ ID NO: 46 - protein, 10 aa
GTRSRKREIL

SEQ ID NO: 47 - protein, 10 aa
SSVQKPEENI

SEQ ID NO: 48 - protein, 10 aa
DSEKEVPSDV

SEQ ID NO: 49 - protein, 9 aa
YSPLPPKVL

SEQ ID NO: 50 - protein, 9 aa
ESDNKYKIA

SEQ ID NO: 51 - protein, 9 aa
ATPYAGEPA

SEQ ID NO: 52 - protein, 10 aa
ETLGEEDKDL

(19) United States
(12) Patent Application Publication
(10) Pub. No.: US 2002/0136733 A1
(43) Pub. Date: Sep. 26, 2002

(54) Malaria peptides

(76) Inventors: Adrian Vivian Sinton Hill, Oxford (GB); Michael Aidoo, Oxford (GB); Catherine Elizabeth Margaret Allsopp, Oxford (GB); Ajit Lalvani, Oxford (GB); Magdalena Plebanski, Oxford (GB); Hilton Carter Whittle, Banjul (GB)

Correspondence Address: WENDEROTH, LIND & PONACK, L.L.P., 2033 K STREET N. W., SUITE 800, WASHINGTON, DC, 20006-1021, US

(21) Appl. No.: 10/042,202

(22) Filed: Jan. 11, 2002

Related U.S. Application Data
(63) Continuation of application No. 08/714,175, filed on Jan. 28, 1997.

(30) Foreign Application Priority Data
Mar. 31, 1994 (GB) 9406492.0

(51) Int. Cl. A61K 39/00; C07K 14/74

(52) U.S. Cl. 424/185.1; 530/350

(57) ABSTRACT

8-11-Mer peptides are identified, from four Plasmodium falciparum antigens, circumsporozoite protein, thrombospondin-related anonymous protein, spirozoite hepatocyte binding antigen and liver-stage antigen-1, as actual or potential cytotoxic T lymphocyte epitopes for human leucocyte antigen class I molecules HLA-A2, HLA-B8, HA-B7 and HLA-B17. Vaccines for immunization against malaria contain these peptides or oligonucleotides coding for them. A method of inducing primary cytotoxic T lymphocyte responses to a chosen antigen or microorganism comprises incubating lymphocytes ex vivo with the chosen antigen or microorganism in the presence of keyhole limpet haemocyanin.

The identification of peptide epitopes for HLA class I molecules is of importance for several areas of biomedical science. Firstly, such epitopes are the central components of vaccines designed to provide immunity mediated by cytotoxic T lymphocytes (CTL) to infectious microorganisms. This is a consequence of the method whereby cytotoxic T lymphocytes function: they recognise a peptide epitope of 8-11 amino acids in length presented by an HLA class I molecule. Secondly, there is increasing interest in the possibility that neoplastic tumours might be ameliorated or cured by inducing, either in vivo or in vitro, CTL that can recognize tumour-specific peptides on HLA class I molecules. Thirdly, the identification of such epitopes recognised in autoimmune disorders would provide insights into pathogenesis and suggest new specific methods of treating these disorders.

This invention deals firstly with the identification of peptides from the malaria parasite Plasmodium falciparum which we identify as epitopes or potential epitopes for particular HLA class I molecules. A variety of evidence (cited in references 1 and 16) suggests that CTL play a role in immunity to this parasitic infection and disease by acting against the liver-stage of infection. Thus these epitopes will be of value for inclusion in vaccines designed to provide immunity to Plasmodium falciparum. Furthermore, this work identifies for the first time CTL epitopes in the P. falciparum antigen, thrombospondin-related anonymous protein (TRAP²), and thus identifies TRAP, and/or peptides from TRAP, as a useful component of a CTL-inducing vaccine against P. falciparum malaria.

This invention deals secondly with a novel method of identifying cytotoxic T lymphocyte epitopes and of producing CTL lines and clones by the use of a new method of inducing CTL responses in vitro.

### THE INVENTION

In one aspect the invention provides the 8 to 11-mer peptides set out in the table below, being either epitopes or potential epitopes for the stated HLA class I molecules, conservative variants thereof, and longer peptides containing these sequences which are sub-units of the indicated antigens. It is envisaged that these peptides are generally 8 to 100 amino acid residues in length, and that the sequence shown in the table is the main functional epitope present. No per se claim is made to the antigen as a whole, nor to any fragment which constitutes the larger part of the antigen.

Two or more of these peptides may be joined together in sequence. The peptide may have an N- or C-terminus carrying a lipid tail, a modification known to enhance CTL induction in vivo.

Also included are oligonucleotides which code for the stated peptides. The term oligonucleotide is here used to encompass nucleic acid chains of about 24 to about 300 nucleotide residues.

Also included are vaccines comprising peptides or oligonucleotides as defined, for immunisation against malaria.

In another aspect the invention provides a method of inducing primary cytotoxic T lymphocyte responses to a chosen antigen or microorganism, which method comprises incubating lymphocytes ex vivo with the chosen antigen or microorganism in the presence of KLH (keyhole limpet haemocyanin) or any other substance which preferentially stimulates a CD45RA⁺ subset of T lymphocyte.

In this method the KLH or other adjuvant is used at a concentration suitable to stimulate the desired CTL response, which optimum concentration may readily be determined by experiment. Preferably IL-7 (interleukin-7) and/or IL-2 (interleukin-2) is also present during the said incubation.

Section A

Identification of P. falciparum Peptides and Epitopes

We have recently described a novel approach to identifying CTL epitopes and potential CTL epitopes in P. falciparum¹. This consisted, in brief, of 1) determining a motif for peptides bound to a particular KLA class I molecules, 2) synthesizing peptides from P. falciparum antigens congruent with this motif, 3) testing whether these peptides bind to that HLA class I allele using a binding assay known as a HLA assembly assay, and 4) testing whether lymphocytes from individuals exposed to malaria could recognise these peptides as epitopes after suitable in vitro restimulation and culture. In that work we identified peptide epitopes and potential peptide epitopes for two HLA class I molecules, HLA-B53 and HLA-B35. Here we extend that work to four further HLA class I molecules: HLA-A2, HLA-B8, HLA-B7 and HLA-B17. 52 peptides are identified that are shown either to be epitopes or potential epitopes for these HLA class I molecules.

### DETAILED DESCRIPTION

Motifs for Eluted Peptides.

The peptide binding motifs of HLA-A2, HLA-B7 and HLA-B8 have been described. For HLA-A2 strong preferences were found at positions 2 and 9 of bound peptides: for leucine, isoleucine and methionine at position 2, and for valine, leucine and isoleucine at position 9^{3, 4} For HLA-B7 the preferred residues were proline at position 2 and a hydrophobic residue at position 9. Leucine, isoleucine, valine, alanine, phenylalanine and tryptophan were the hydrophobic residues preferred⁵. For HLA-B8 preferred residues were found at positions 3, 5 and 9⁶. At positions 3 and 5 lysine or arginine are preferred and at position 9, leucine or isoleucine or valine. The motif for peptides bound to HLA-B17 has not been reported so we determined this exactly as described for HLA-B35¹, using instead of the cell line CIR-B35, the cell line CIR-B58. Hence the peptide motif determined is strictly for HLA-B58 which along with HLA-B57 constitutes a subtype of HLA-B17. The primary amino acid sequences of HLA-B57 and HLA-B58 are very similar indicating that the types of peptides bound are likely to be very similar, at least in their key anchor residues. Hence we refer to the motif determined for CIR-B58 as a motif for HLA-B17. For HLA-B17, preferred amino acids were observed at positions 2 and 9: serine and threonine were preferred at position 2 and a hydrophobic residue (defined as for B7 above) at position 9.

Peptide Synthesis and Binding Assays

As described previously for identification of HLA-B35 and HLA-B53 epitopes¹, peptides were synthesised to correspond to these four motifs from the primary amino acid sequences of four P. falciparum pre-erythrocytic antigens: CSP, TRAP, LSA-1 and SHEBA. Over one hundred peptides were synthesized. Binding assays⁷ were performed on selected peptides to determine whether they bound to HLA-A2, -B7, -B8, or -B58 using the untransfected T2 cell line (for HLA-A2) or the T2 cell line transfected with HLA-B7 (used to assess binding to HLA-B7s), or with HLA-B8 (used to assess binding to HLA-B8), or with HLA-B58 (used to assess binding to HLA-B58).

Cytotoxic T Lymphocyte Assays

Peptides shown to bind to a particular HLA class I allele were tested for CTL recognition in assays using lymphocytes from malaria-exposed Gambians, as described previously¹. A minority of peptides, synthesized to correspond to a peptides binding motif were not tested in the relevant assembly assay but tested only for CTL recognition. Cells from 82 adult Gambians and 53 Gambian children, all exposed to malaria, were used in the course of these studies. The children and adults were HLA typed using cellular or molecular techniques as described previously¹. Peptides were incubated with cells either singly, or in pools as described¹ at concentrations of 10-100 μM. Peptides could either be left with the cells for the duration of restimulation (as reported¹) or washed off after an hour. Cells were cultured for 1-3 weeks before a standard CTL chromium release assay¹ was performed using HLA matched or autologous B cell line targets pre-pulsed with the peptide to be tested. Peptides were tested either singly or in pools for CTL recognition. 10% or greater was regarded as a significant level of specific lysis.

Peptides Identified: CTL Epitopes.

The sequences of all peptides referred to are shown in the table.

i) HLA-A2. Two Gambian individuals showed positive CTL responses to HLA-A2 peptides. One individual (Z62) recognised a pool of three TRAP peptides, tr26, tr29 and tr39. Insufficient cells were available to determine which of these three peptides was the epitope. However a second adult (Z60) also recognised this pool of three peptides and subsequently showed significant lysis of tr39- but not tr26-, nor tr29-pulsed target cells. Hence tr39 is an HLA-A2-restricted epitope. Both individuals were of the HLA-A*0201 subtype of HLA-A2. All three of these peptides bound to HLA-A2 in the assembly assay.

Further studies consisting of cytotoxic T lymphocyte assays using lymphocytes from malaria-exposed Gambians have confirmed that the following peptides listed in the table are cytotoxic T lymphocyte epitopes. For individuals with HLA-A2, CTL from at least one individual have been found to recognise the peptides tr26, tr29, cp36, cp37, cp38 and cp39. These epitopes for HLA-A2 are in addition to tr39 identified previously.

ii) HLB7. CTL from one child with HLA-B7 showed significant lysis of HLA-B7 matched target cells pre-pulsed with the peptide pool cp6, cp6.1 and cp6.2. These peptides are encoded by allelic variants of the same region of the circumsporozoite protein gene⁸ identifying this sequence as an HLA-B7 restricted epitope. All three peptides bound to HLA-B7 in the HLA assembly assay. CTL from one adult (Z174) also recognised a pool of four peptides containing cp6, cp6.1, cp6.2 and cp21.

iii) HLA-B8. CTL from one adult (Z42) showed significant lysis of a pool of peptides containing tr42, tr43, tr44 and tr45. Two of these four peptides, tr42 and tr43 bound to HLA-B8 in the HLA assembly assay. Subsequently the same CTL line showed specific lysis of tr42 only defining this as an HLA-B8-restricted CTL epitope. CTL from another adult (Z130) showed specific lysis of the tr43 peptide, identifying this as another CTL epitope for HLA-B8. The tr42 and tr43 epitopes overlap. Two other adults (Z80 and Z169) showed significant lysis of target cells pre-pulsed with a pool of four CSP peptides, cp43, cp44, cp45 and cp46. Only one of these four, cp43, bound to HLA-B8 in the HLA assembly assay, identifying it as an HLA-B8-restricted CTL epitope.

iv) HLA-B17. CTL from one adult showed 18% specific lysis of a pool of TRAP peptides (tr49-64) after two weeks of cell culture. One week later significant lysis was observed against two sub-pools of peptides: tr57, tr58, tr59, tr60 constituting one sub-pool, and tr61, tr62, tr63 and tr64 constituting the other subpool. These data indicate the presence of one CTL epitope in each of these TRAP peptide sub-pools. Two adults (Z61 and Z63) showed significant lysis of a large pool of peptides from liver stage antigen (LSA-1), indicating the presence of at leas one CTL epitope amongst these peptides. This pool contained the following 13 peptides: ls36, ls37, ls39, ls40, ls42, ls45, ls48, ls49, ls50, ls51, ls53, ls54, ls55.

Further Peptides.

In addition to these epitopes identified above further peptides were identified as “potential epitopes” as follows. The designation “potential epitope” is justified as follows. All of these peptides are from antigens shown to be targets of CTL recognition. They are furthermore capable of binding with high affinity to the specified HLA class I molecule. Although they have not been shown to be recognised by CTL grown from malaria exposed individuals their ability to bind to the relevant class I molecule indicates that they are likely to be presented on the surface of malaria-infected hepatocytes in vivo. Hence, even if detectable levels of CTL to these peptides are not found in malaria-exposed individuals, induction of CTL to these peptides may be a useful means of immunizing against P. falciparum malaria.

i) HLA-A2. The following peptides were shown to bind to HLA-A2 in the HLA assembly assay and are therefore potential CTL epitopes: ls10, ls11, ls19, ls23. The peptide cp36 was also capable of inducing a primary CTL response in malaria-unexposed individuals (see below).

ii) HLA-B7. The following peptides were found to bind to HLA-B7 in the HUA assembly assay and are therefore potential epitopes: cp21, ls6, tr6, tr13, tr15, tr21, sh1, sh6.

iii) HLA-B17. The following peptides were found to bind to HLA-B17 in the HLA assembly assay and are therefore potential epitopes: cp48, cp55, cp56, and all the 13 ls peptides listed above as being present in the pool recognised by two individuals with HLA-B17.

Further evidence for the potential usefulness of the peptides tr43, cp36 and variants of cp36 (cp37-39) in a malaria vaccine is presented below where we show that CTL may be generated in vitro against these peptides from malaria un-exposed individuals.

This work identifies TRAP as a P. falciparum antigen which induces cytotoxic T lymphocyte responses in individuals exposed to endemic malaria. TRAP is expressed on sporozoites⁹ as well as blood-stage malaria parasite and will therefore be present in the infected liver cell. We show here that TRAP contains CTL epitopes for three very common HLA class I antigens (HLA-A2, -B8 and -B17) and therefore the induction of CTL to TRAP may be an important requirement of an effective CTL-inducing vaccine against P. falciparum.

As there is evidence that better CTL responses may be induced in vivo using epitopes or epitopes with a limited amount of flanking sequence than by using the whole antigen, the epitopes used here may be particularly valuable for the induction of CTL responses in vivo¹⁵.

This specification identifies for the first time the existence of CTL responses to the antigen TRAP in humans exposed to P. falciparum parasites. It is known from studies of rodent malaria and indirectly from studies of human P. falciparum malaria¹⁶ that CTL are likely to play a protective role but the target antigens of these CTL have been unclear. By identifying TRAP as a target of CTL responses in humans we identify it as a favourable antigen for inclusion in a vaccine designed to induce protective CTL responses. Moreover, we show here that TRAP contains conserved CTL epitopes for the very common class I antigen, HLA-A2, which is the most prevalent HLA-A or -B molecule in Caucasians, making TRAP of particular importance for immunization through CTL of Caucasian populations.

There are several means by which the CTL epitopes identified here may be used to stimulate an immune response in vivo in humans. Either the peptides, or longer peptides containing them, can be used alone or with an adjuvant, such as incomplete Freund's adjuvant¹⁷ or QS-21¹⁸ or NAGO¹⁹ or AF²⁰, or as peptides with a lipid-tail added²¹, a means that has been shown to enhance CTL induction in vivo. Alternatively, the epitopes can be delivered by recombining nucleotides encoding them into a gene coding for a particle such as a recombinant Ty-virus-like-particle²² or a recombinant hepatitis B virus antigen particle²³. Alternatively, nucleotides encoding these epitopes can be incorporated into a recombinant virus such as a vaccinia virus or an attenuated vaccinia virus²⁴. Another means is to generate a recombinant bacterium such as a recombinant Salmonella containing nucleotides encoding these epitopes²⁵. Another means is to incorporate nucleotides encoding the epitopes identified into an expression vector, such as a DNA vaccine²⁶, that can express these epitopes after immunization. Finally, ribonucleotides coding for these epitopes can be used as an RNA-based vaccine²⁷ to express these epitopes in vivo.

Section B

A Method of Inducing Primary CTL Responses In Vitro

Although secondary (or recall) CTL responses to a variety of infectious micro-organisms can now be detected (e.g. as described for malaria above), CTL cannot be grown in this way from individuals unexposed to antigen or microorganism (ref.1 and unpublished data). We describe here a novel method of growing “primary” CTL i.e. from previously unprimed individuals. This method can be employed for generating cell lines and clones which may be useful in various ways: to identify potential epitopes amongst a pool of peptides which bind to an HLA class I molecule; to identify peptides presented by HLA molecules on the surface of a cell using a CTL assay; for in vivo therapeutic use for the treatment of infectious or neoplastic disease. We demonstrate this method by describing the generation of CTL lines and clones to two peptides from P. falciparum from the lymphocytes of three individuals who have not been exposed to or infected by this parasite.

The method described here for inducing primary CTL responses in vitro may be particularly useful in cancer immunotherapy. Studies in mice have demonstrated the potential of therapy with ex vivo cultured CTL²⁸, and human tumor-specific CTL have been identified in the peripheral blood or tumor-infiltrating lymphocytes from patients with melanoma and renal cell carcinoma^{29, 30}. Additionally, induction of CTL against viral antigen epitopes in vitro may be useful in the therapy of viral infections such as HIV³¹.

### DETAILED DESCRIPTION

Peripheral blood mononuclear cells from individuals never exposed to malaria were separated on Ficoll-hypaque and prepulsed for 2 hours with 20-100 μM peptide. In the case of two HLA-A2 individuals the peptide was cp36; in the case of one HLA-B8 individual the peptide was tr43. The cells were then washed once and 5 million cells were incubated in a 2 ml well (in a standard humidified incubator with 5% CO₂) in α-MEM (minimal essential medium, GIBCO, UK) with autologous heat-inactivated human serum and 2 μg per ml of keyhole limpet haemocyanin (KLH, Calbiochem, California, USA). The addition of the latter was based on our previous showing that this preferentially stimulates the CD45RA⁺ (naive) subset of CD4 T lymphocytes^{10, 11}. This CD4 T lymphocyte subset has been shown previously to promote CD8 T cell activity¹².

After 72 hours interleukin-2 (Cetus, California, USA) was added at a concentration of 10 units per ml and the cells were cultured for a further 4 days. Then 5000 of the cells were restimulated in 150 μl of α-MEM with 10% autologous human serum with 100,000 irradiated autologous peripheral blood lymphocytes, that had been pre-pulsed for one hour in 20 μM of the same peptide used for the first stimulation and then washed once. On the following day 3 units of IL-2 was added to the well and the incubation continued for 8 further days. Then standard CTL assays were performed at an effector to target ratio of 5-10:1. Peptide specific lysis in excess of 10% was observed in from 5-15% of wells. This CTL activity was peptide-specific and dependent on CD8⁺ lymphocytes. In the case of cp36 the CTL responses were shown to be HLA-A2.1 restricted. In the case of peptide tr43 the CTL responses were HLA-B8 restricted.

TABLE

| | Sequence | | | | | | | | | | |
|---|---|---|---|---|---|---|---|---|---|---|---|
| label | 1 | 2 | 3 | 4 | 5 | 6 | 7 | 8 | 9 | 10 | Position |
| HLA-A2 | | | | | | | | | | | |
| tr26 * | H | L | G | N | V | K | Y | L | V | | 3 |
| tr29 | L | L | M | D | C | S | G | S | I | | 51 |
| tr39 * | G | I | A | G | G | L | A | L | L | | 500 |
| ls10 | I | L | Y | I | S | F | Y | F | I | | 4 |
| ls11 | Y | I | S | F | Y | F | I | L | V | | 6 |
| ls19 | G | I | Y | K | E | L | E | D | L | | 1801 |
| ls23 | H | I | F | D | G | D | N | E | I | | 1883 |
| cp36 * | Y | L | K | T | I | Q | N | S | L | | 334 |
| cp37 * | Y | L | Q | K | I | Q | N | S | L | | 334 |
| cp38 * | Y | L | Q | K | I | K | N | S | L | | 334 |
| cp39 * | Y | L | N | K | I | Q | N | S | L | | 334 |
| HLA-B8 | | | | | | | | | | | |
| cp43 | L | R | K | P | K | H | K | K | L | | 134 |
| cp44 | L | K | K | I | K | N | S | I | S | | 335 |
| cp45 | Q | V | R | I | K | P | G | S | A | | 358 |
| cp46 | A | N | K | P | K | D | G | L | D | | 366 |
| tr42 * | A | S | K | N | K | E | K | A | L | | 107 |
| tr43 * | K | N | K | E | K | A | L | I | I | | 109 |
| HLA-B7 | | | | | | | | | | | |
| cp6 * | K | P | N | D | P | N | R | N | V | | 300 |
| cp6.1 * | M | P | N | Y | P | N | R | N | V | | 300 |
| cp6.2 * | M | P | N | N | P | N | R | N | V | | 300 |
| ls6 | K | P | I | V | Q | Y | D | N | F | | 1786 |
| sh1 | I | P | S | L | A | L | M | L | I | | 7 |
| sh6 | M | P | L | E | T | Q | L | A | I | | 77 |
| cp21 | N | P | D | P | N | A | N | P | N | V | 120 |
| tr6 | N | P | E | N | P | P | N | P | D | I | 348 |
| tr13 | I | P | D | S | I | Q | D | S | L | | 164 |
| tr15 | E | P | A | P | F | D | E | T | L | | 529 |
| tr21 | G | P | F | M | K | A | V | C | V | | 228 |
| HLA-B17 | | | | | | | | | | | |
| cp48 | L | S | V | S | S | F | L | F | V | | 8 |
| cp55 | G | S | A | N | K | P | K | D | E | L | 364 |
| cp56 | C | S | S | V | F | N | V | V | | | 388 |
| ls36 | N | S | E | K | D | E | I | I | | | 28 |
| ls37 | G | S | S | N | S | R | N | R | I | | 42 |
| ls39 | V | S | Q | T | N | F | K | S | L | | 92 |
| ls40 | K | S | L | L | R | N | L | G | V | | 98 |
| ls42 | Q | S | D | S | E | Q | E | R | L | | 179 |
| ls45 | R | T | K | A | S | K | E | T | L | | 1187 |
| ls48 | H | T | L | E | T | V | N | I | | | 1742 |
| ls49 | I | S | D | V | N | D | F | Q | I | | 1749 |
| ls50 | I | S | K | Y | E | D | E | I | | | 1757 |
| ls51 | I | S | A | E | Y | D | D | S | L | | 1764 |
| ls53 | K | S | L | Y | D | E | H | I | | | 1854 |
| ls54 | L | S | E | D | I | T | K | Y | F | | 1898 |
| ls55 | T | K | Y | F | M | K | L | | | | 1902 |
| tr57 | K | T | A | S | C | G | V | W | D | EW | 240 |
| tr58 | G | T | R | S | R | K | R | E | I | L | 260 |
| tr59 | S | S | V | Q | K | P | E | E | N | I | 311 |
| tr60 | D | S | E | K | E | V | P | S | D | V | 367 |
| tr61 | Y | S | P | L | P | P | K | V | L | | 415 |
| tr62 | E | S | D | N | K | Y | K | I | A | | 490 |
| tr63 | A | T | P | Y | A | G | E | P | A | | 523 |
| tr64 | E | T | L | G | E | E | D | K | D | L | 535 |

*Peptide identified as an epitope for a secondary cytotoxic T lymphocyte response.

Table: Peptides from four Plasmodium falciparum antigens, circumsporozoite protein (cp), thrombospondin-related anonymous protein (tr) spirozoite hepatocyte binding antigen (sh) and liver-stage antigen-1 (ls), that are here identified as CTL epitopes or as potential CTL epitopes for particular HLA class I molecules. Epitopes are shown in bold type. The position of the first amino acid of the peptide in the published amino acid sequence (CSP—ref. 13; LSA-1 —ref. 14; TRAP—ref. 2) is shown. Note that tr57 is 11 amino acids. in length. The standard one letter amino acid code is used.

### REFERENCES

1. Hill, A. V. S. et al. Nature 360, 434-439 (1992).

2. Robson, K. J. H. et al. Nature 335, 79-82 (1988).

3. Falk, K., Rotzscke, O., Stevanovic, S., Jung, G. & Rammensee, H. Nature 351, 290-296 (1991).

4. Hunt, D. F. et al. Science 255, 1261-6 (1992)

5. Huczko, E. L. et al. J. Immunol. 151, 2572-2587 (1993)

6. Sutton, J. et al. Eur. J. Immunol. 23, 447-453 (1993).

7. Elvin, J. et al. J. Immunol. Methods 158, 161-171 (1992)

8. Doolan, D. A., Saul, A. & Good, M. F. Infection and Immunity 60, 675-682(1992)

9. Cowan, G., Krishna, S., Crisanti, A. & Robson, K. J. H. Lancet 339, 1412-1413

10. Plebanski, M., Saunders, M., Burtles, S. S., Crowe, S. & Hooper, D. C. Immunology 75: 86-92 (1992).

11. Plebanski, M. & Burtles, S. S. J. Immunol. Methods In Press (1994).

12. Morimoto, C. et al. Eur. J. Immunol. 16, 198-204 (1986).

13. Dame, J. B. et al. Science 225, 593-599 (1984).

14. Zhu, J. & Hollingdale, M. Mol. Biochem. Parasitol. 48, 223-226 (1991).

15. Lawson, C. M. et al. J. Virol. 68: 3505-11 (1994).

16. Lalvani, A, et al. Research in Immunology 145: 461-468 (1994).

17. Kast, W. M. et al. Proc Natl Acad Sci USA 88: 2282-2287 (1991).

18. Newman, M. J. et al. J. Immunol. 148: 2357-64 (1992).

19. Zheng, B. et al. Science 256: 1560-1563 (1992).

20. Raychaudhuri et al. Proc Natl Acad Sci USA 89: 8308-8312 (1992).

21. Deres, K. et al. Nature 342: 561-3 (1989).

22. Layton, G. T. et al. J. Immunol. 151: 1097-1107 (1993).

23. Tindle, R. W. et al. Virology 200: 547-557 (1994).

24. Cox, W. I. et al. Virology 195: 845-850 (1993).

25. Chatfield, S. N. et al. Vaccine 10: 53-60 (1992).

26. Ulmer, J. B. et al. Science 259: 1745-49 (1993).

27. Martinon, F. et al. Eur. J. Immunol. 23: 1719-22 (1993).

28. Greenberg, P. D. Adv. Immunol. 49: 281 (1991).

29. Cerottini, J. C. et al. Ann. Oncol. 3: 11 (1992).

30. Koo, A. S. et al. J. Immunotherapy 10: 347 (1991).

31. Riddell, S. R. & Greenberg, P. D. Curr. Top. Microbiol. Immunol. 189: 9-34 (1994).

1. The peptides, being either epitopes or potential epitopes for the stated HLA (human leucocyte antigen) class I molecules, conservative variants thereof, and longer peptides containing these sequences which are sub-units of the indicated antigens

| | Sequence | | | | | | | | | | |
|---|---|---|---|---|---|---|---|---|---|---|---|
| label | 1 | 2 | 3 | 4 | 5 | 6 | 7 | 8 | 9 | 10 | Position |
| HLA-A2 | | | | | | | | | | | |
| tr26 | H | L | G | N | V | K | Y | L | V | | 3 |
| tr29 | L | L | M | D | C | S | G | S | I | | 51 |
| tr39 | G | I | A | G | G | L | A | L | L | | 500 |
| ls10 | I | L | Y | I | S | F | Y | F | I | | 4 |
| ls11 | Y | I | S | F | Y | F | I | L | V | | 6 |
| ls19 | G | I | Y | K | E | L | E | D | L | | 1801 |
| ls23 | H | I | F | D | G | D | N | E | I | | 1883 |
| cp36 | Y | L | K | T | I | Q | N | S | L | | 334 |
| cp37 | Y | L | Q | K | I | Q | N | S | L | | 334 |
| cp38 | Y | L | Q | K | I | K | N | S | L | | 334 |
| cp39 | Y | L | N | K | I | Q | N | S | L | | 334 |
| HLA-B8 | | | | | | | | | | | |
| cp43 | L | R | K | P | K | H | K | K | L | | 134 |
| cp44 | L | K | K | I | K | N | S | I | S | | 335 |
| cp45 | Q | V | R | I | K | P | G | S | A | | 358 |
| cp46 | A | N | K | P | K | D | G | L | D | | 366 |
| tr42 | A | S | K | N | K | E | K | A | L | | 107 |
| tr43 | K | N | K | E | K | A | L | I | I | | 109 |
| HLA-B17 | | | | | | | | | | | |
| cp48 | L | S | V | S | S | F | L | F | V | | 8 |
| cp55 | G | S | A | N | K | P | K | D | E | L | 364 |
| cp56 | C | S | S | V | F | N | V | V | | | 388 |
| ls36 | N | S | E | K | D | E | I | I | | | 28 |
| ls37 | G | S | S | N | S | R | N | R | I | | 42 |
| ls39 | V | S | Q | T | N | F | K | S | L | | 92 |
| ls40 | K | S | L | L | R | N | L | G | V | | 98 |
| ls42 | Q | S | D | S | E | Q | E | R | L | | 179 |
| ls45 | R | T | K | A | S | K | E | T | L | | 1187 |
| ls48 | H | T | L | E | T | V | N | I | | | 1742 |
| ls49 | I | S | D | V | N | D | F | Q | I | | 1749 |
| ls50 | I | S | K | Y | E | D | E | I | | | 1757 |
| ls51 | I | S | A | E | Y | D | D | S | L | | 1764 |
| ls53 | K | S | L | Y | D | E | H | I | | | 1854 |
| ls54 | L | S | E | D | I | T | K | Y | F | | 1898 |
| ls55 | T | K | Y | F | M | K | L | | | | 1902 |
| tr57 | K | T | A | S | C | G | V | W | D | EW | 240 |
| tr58 | G | T | R | S | R | K | R | E | I | L | 260 |
| tr59 | S | S | V | Q | K | P | E | E | N | I | 311 |
| tr60 | D | S | E | K | E | V | P | S | D | V | 367 |
| tr61 | y | S | P | L | P | P | K | V | L | | 415 |
| tr62 | E | S | D | N | K | Y | K | I | A | | 490 |
| tr63 | A | T | P | Y | A | G | E | P | A | | 523 |
| tr64 | E | T | L | G | E | E | D | K | D | L | 535 |

these peptides being selected from three Plasmodium falciparum antigens, circumsporozoite protein (cp), thrombospondin-related anonymous protein (tr) and liver-stage antigen-1 (ls),

2. A peptide comprising at least two of the sequences listed in claim 1.

3. A peptide as claimed in claim 1 or claim 2 having an N-terminus or C-terminus carrying a lipid tail.

4. A peptide as claimed in any one of claims 1 to 3, comprising 8-100 amino acid residues.

5. A vaccine comprising at least one peptide according to any one of claims 1 to 4, for immunisation against malaria.

6. Use of Plasmodium falciparum gene or protein TRAP (thrombospondin-related anonymous protein) as a cytotoxic T lymphocyte-inducing gene or protein for immunization against malaria.

7. oligonucleotides which code for the peptides claimed in any one of claims 1 to 4.

8. A vaccine comprising at least one oligonucleotide according to claim 7 for expression in vivo for immunization against malaria.

9. A method of inducing primary cytotoxic T lymphocyte responses to a chosen antigen or microorganism, which method comprises incubating lymphocytes ex vivo with the chosen antigen or microorganism in the presence of KLH (keyhole limpet haemocyanin) or any other substance which preferentially stimulates a CD45RA⁺ subset of T lymphocyte.

10. A method as claimed in claim 9, wherein IL-7 (interleukin-7) and/or IL-2 (interleukin-2) is also present during incubation.

11. Use of any one of the peptides:

| | Sequence | | | | | | | | | | |
|---|---|---|---|---|---|---|---|---|---|---|---|
| label | 1 | 2 | 3 | 4 | 5 | 6 | 7 | 8 | 9 | 10 | Position |
| HLA-B7 | | | | | | | | | | | |
| cp6 | M | P | N | D | P | N | R | N | V | | 300 |
| cp6.1 | M | P | K | Y | P | N | R | N | V | | 300 |
| cp6.2 | M | P | N | N | P | N | R | N | V | | 300 |
| ls6 | K | P | I | V | Q | Y | D | N | F | | 1786 |
| sh1 | I | P | S | L | A | L | M | L | I | | 7 |
| sh6 | M | P | L | E | T | Q | L | A | I | | 77 |
| cp21 | N | P | D | P | N | A | N | P | N | V | 120 |
| tr6 | N | P | E | N | P | P | N | P | D | I | 348 |
| tr13 | I | P | D | S | I | Q | D | S | L | | 164 |
| tr15 | E | P | A | P | F | D | E | T | L | | 529 |
| tr21 | G | P | F | M | K | A | V | C | V | | 228 |

and conservative variants thereof and longer peptides containing the sequences which are sub-units of the stated antigen, and of oligonucleotides which code for the said peptides, as a cytotoxic T lymphocyte-inducer for immunization against malaria of individuals possessing a HLA-B7 allele.
